(19) United States
(12) Patent Application Publication
(10) Pub. No.: US 2024/0307569 A1
(43) Pub. Date: Sep. 19, 2024

(54) Cartridge Loader

(71) Applicant: Northstar Medical Technologies, LLC, Beloit, WI (US)

(72) Inventors: Michael ALLEX, Verona, WI (US); Dorian LUST, Columbus, WI (US)

(73) Assignee: Northstar Medical Technologies, LLC, Beloit, WI (US)

(21) Appl. No.: 18/223,198

(22) Filed: Jul. 18, 2023

Related U.S. Application Data
(60) Provisional application No. 63/390,492, filed on Jul. 19, 2022.

(51) Int. Cl. B65B 67/02; A61L 2/10; A61L 2/26; G21F 5/015

(52) U.S. Cl. CPC B65B 67/02; A61L 2/10; A61L 2/26; G21F 5/015; A61L 2202/11; B65B 2210/06

(57) ABSTRACT

A cartridge loader is provided. The cartridge loader can include a loader body, a top loader bay slidingly engaged to the loader body, and a bottom loader bay slidingly engaged to the loader body. The cartridge loader can further include a cleaning assembly coupled to the loader body. The cartridge loader can be manipulated from a first configuration to a second configuration, such that when in the first configuration, the cartridge loader can receive a cartridge shield containing a cartridge therein, and when in the second configuration, if no cartridge shield is received, the cleaning assembly is configured to disinfect the cartridge loader.

### CROSS-REFERENCE TO RELATED APPLICATION

This application claims the benefit of the filing date of U.S. Provisional Application Ser. No. 63/390,492, filed Jul. 19, 2022, entitled, “Cartridge Loader”, which is hereby incorporated by reference as if fully set forth herein.

### TECHNICAL FIELD

This disclosure generally relates to a cartridge loader. More particularly, this disclosure relates to a loader for disposable cartridges containing radioactive materials.

### BACKGROUND

During production of radioisotopes, elution may be carried out in order to separate a product radioisotope. For example, Technetium-99m (^{99m}Tc)—a commonly used medical radioisotope—may be separated and eluted from Molybdenum-99 (Mo-99).

Disposable cartridges-such as separation cartridges and product cartridges—can be used to radionuclide of different stages, such as a parent radionuclide and a daughter radionuclide. A process of separating daughter radionuclide from a parent radionuclide using cartridges is described in U.S. patent application Ser. No. 14/528,789, titled “Separator Cartridge for Radionuclide”, which is hereby incorporated by reference in full.

However, disposable cartridges must be replaced between elution that requires intervention by a user, which can increase the potential radiation exposure to the user. Thus, there is a need for a cartridge loading system that reduces a user's proximity to and potential contact with radioactive materials.

### BRIEF SUMMARY

An aspect of this disclosure pertains to a cartridge loader that reduces a user's proximity to and potential contact with radioactive materials through improved shielding and cartridge loading mechanisms.

A first aspect of this disclosure pertains to a cartridge loader including a loader body; a top loader bay slidingly engaged to the loader body; and a bottom loader bay slidingly engaged to the loader body, wherein the loader body is configured to receive a cartridge shield with a cartridge provided therein between the top loader bay and the bottom loader bay, and wherein the cartridge loader can be manipulated between a first configuration and a second configuration.

A second aspect of this disclosure pertains to the cartridge loader of the first aspect further including a handle track provided on the loader body; and a handle slidingly engaging the handle track such that the handle is configured to move in both a lateral direction and a vertical direction with respect to the loader body.

A third aspect of this disclosure pertains to the cartridge loader of the second aspect, wherein the handle track further comprising a horizontal portion and a vertical portion.

A fourth aspect of this disclosure pertains to the cartridge loader of the second aspect, wherein the top loader bay further comprising a first inner component and a first outer component, wherein the first inner component is slidingly received within a first bore of the first outer component.

A fifth aspect of this disclosure pertains to the cartridge loader of the fourth aspect, wherein the first inner component further comprising a top protrusion configured to be inserted into a first end of the cartridge when the cartridge loader is in the second configuration.

A sixth aspect of this disclosure pertains to the cartridge loader of the fifth aspect, wherein the top protrusion is removed from the cartridge when the cartridge loader is in the first configuration.

A seventh aspect of this disclosure pertains to the cartridge loader of the fourth aspect, wherein the first outer component further comprising a channel such that the first inner component can be coupled to a first slide through the channel.

An eighth aspect of this disclosure pertains to the cartridge loader of the seventh aspect, wherein the first outer component is coupled to the handle such that the first outer component can be lifted away from the bottom loader bay when the cartridge loader is in the first configuration, and the first outer component can be pushed toward the bottom loader bay when the cartridge loader is in the second configuration.

A nineth aspect of this disclosure pertains to the cartridge loader of the fourth aspect, wherein the first outer component further comprising a first receiving portion for receiving a portion of the cartridge shield.

A tenth aspect of this disclosure pertains to the cartridge loader of the first aspect, wherein the top loader bay further comprising a magnet for holding the cartridge shield.

An eleventh aspect of this disclosure pertains to the cartridge loader of the fourth aspect, wherein the bottom loader bay further comprising a second inner component and a second outer component, wherein the second inner component is slidingly received within a second bore of the second outer component.

A twelfth aspect of this disclosure pertains to the cartridge loader of the eleventh aspect, wherein the second inner component further comprising a bottom protrusion configured to be inserted into a second end of the cartridge when the cartridge loader is in the second configuration.

A thirteenth aspect of this disclosure pertains to the cartridge loader of the twelfth aspect, wherein the bottom protrusion is removed from the cartridge when the cartridge loader is in the first configuration.

A fourteenth aspect of this disclosure pertains to the cartridge loader of the eleventh aspect, wherein the second outer component further comprising a channel such that the second inner component can be coupled to a second slide through the channel.

A fifteenth aspect of this disclosure pertains to the cartridge loader of the eleventh aspect, wherein the second outer component further comprising a second receiving portion for receiving a portion of the cartridge shield.

A sixteenth aspect of this disclosure pertains to the cartridge loader of the first aspect, wherein the bottom loader bay further comprising a magnet for holding the cartridge shield.

A seventeenth aspect of this disclosure pertains to the cartridge loader of the first aspect further including a cleaning assembly slidingly coupled to the loader body, wherein when the cartridge loader is in the second configuration without receiving the cartridge shield, the cleaning assembly is configured to disinfect the top loader bay and the bottom loader bay.

An eighteenth aspect of this disclosure pertains to the cartridge loader of the seventeenth aspect, wherein the cleaning assembly further comprising one or more ultraviolet light-emitting diodes (UV LEDs) positioned to disinfect the top loader bay and the bottom loader bay.

A nineteenth aspect of this disclosure pertains to the cartridge loader of the first aspect, wherein the bottom loader bay is further configured to hold a vial shield with a vial provided therein.

A twentieth aspect of this disclosure pertains to the cartridge loader of the first aspect, wherein the bottom loader bay is configured to slide between a first position and a second position due to friction caused by the cartridge shield as the cartridge loader transitions from the first configuration to the second configuration.

### BRIEF DESCRIPTION OF THE DRAWINGS

FIG. 1 illustrates a perspective view of a cartridge shield and a cartridge loader according in a first configuration to an embodiment;

FIG. 2 illustrates a perspective view of the cartridge shield of FIG. 1 loaded into the cartridge loader of FIG. 1;

FIG. 3 illustrates a cut-away view of FIG. 2;

FIG. 4 illustrates the cartridge loader of FIG. 1 in a second configuration according to an embodiment;

FIG. 5 illustrates a cut-away view of FIG. 4;

FIG. 6 illustrates a cut-away view of a cartridge loader in a first configuration with a cleaning assembly in a forward position according to an embodiment;

FIG. 7 illustrates a cut-away view of the cartridge loader of FIG. 6 in a second configuration with the cleaning assembly in a rearward position according to an embodiment;

FIG. 8 illustrates a cut-away view of the cartridge loader of FIG. 6 in a second configuration with the cleaning assembly in a forward position according to an embodiment;

FIG. 9 illustrates an isometric view of FIG. 8;

FIG. 10 illustrates an exploded view of the cartridge shield of FIG. 1;

FIG. 11 illustrates a perspective view of a cartridge separate from the cartridge shield of FIG. 1;

FIG. 12 illustrates a perspective view of a product cartridge shield, a vial shield, and a product cartridge loader in a first configuration according to an embodiment;

FIG. 13 illustrates a perspective view of the product cartridge shield and the vial shield of FIG. 12 loaded into the product cartridge loader of FIG. 12;

FIG. 14 illustrates a cut-away view of FIG. 13;

FIG. 15 illustrates the product cartridge loader of FIG. 12 in a second configuration according to an embodiment;

FIG. 16 illustrates a cut-away view of FIG. 15;

FIG. 17 illustrates an exploded view of the product cartridge shield of FIG. 12; and

FIG. 18 illustrates a perspective view of a product cartridge separate from the product cartridge shield of FIG. 12.

Before explaining the disclosed embodiment of this disclosure in detail, it is to be understood that the invention is not limited in its application to the details of the particular arrangement shown, since the invention is capable of other embodiments. Exemplary embodiments are illustrated in referenced figures of the drawings. It is intended that the embodiments and figures disclosed herein are to be considered illustrative rather than limiting. Also, the terminology used herein is for the purpose of description and not of limitation.

### DETAILED DESCRIPTION

While this invention is susceptible of embodiments in many different forms, there are shown in the drawings and will be described in detail herein specific embodiments with the understanding that the present disclosure is an exemplification of the principles of the invention. It is not intended to limit the invention to the specific illustrated embodiments. The features of the invention disclosed herein in the description, drawings, and claims can be significant, both individually and in any desired combinations, for the operation of the invention in its various embodiments. Features from one embodiment can be used in other embodiments of the invention.

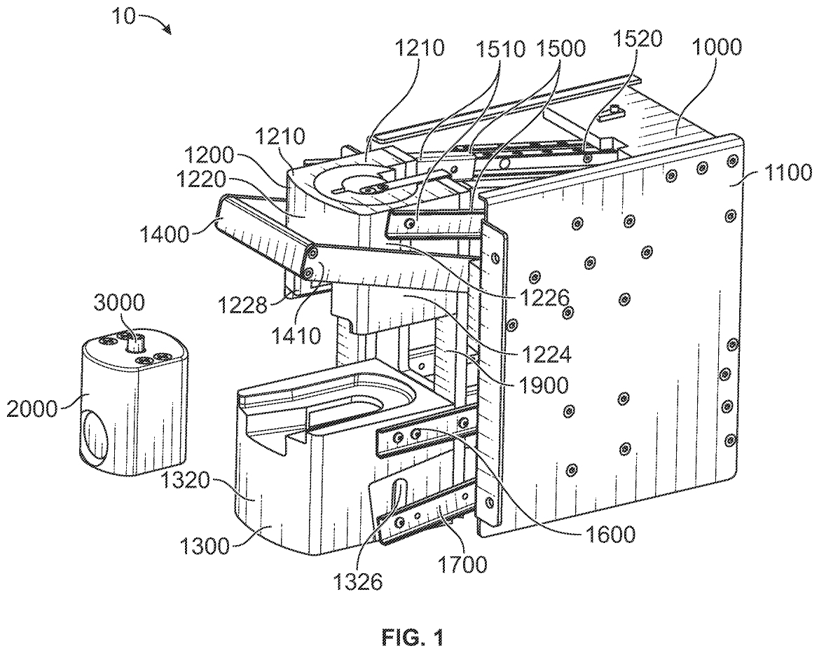

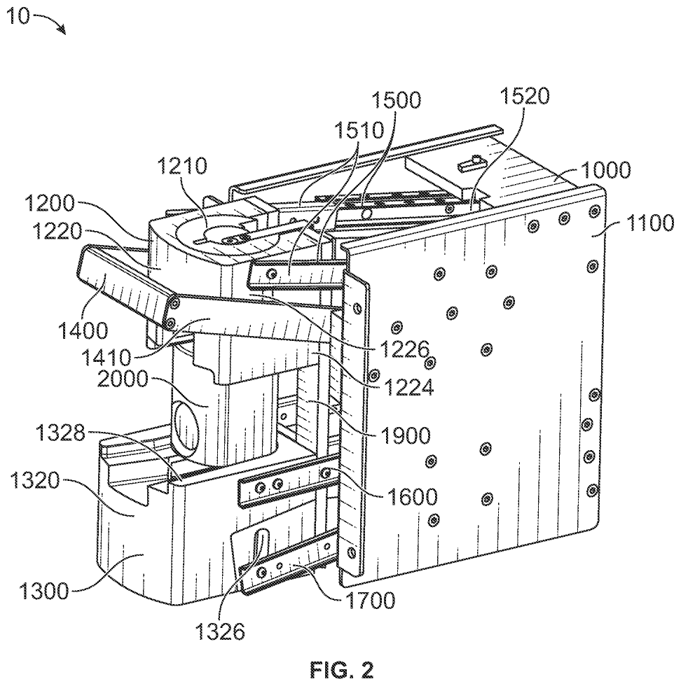

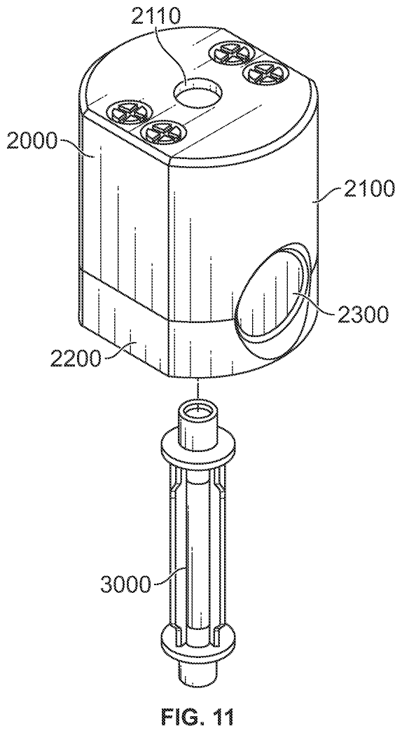

As shown in FIGS. 1-5, embodiments of this disclosure include a system 10 that includes a loader 1000 and a cartridge shield 2000. In some embodiments, the loader 1000 can be used in conjunction with a cartridge 3000 (such as a separation cartridge) held in cartridge shield 2000 (see FIG. 11).

Referring to FIGS. 1-5, the loader 1000 can include a loader body 1100. A top loader bay 1200 and a bottom loader bay 1300 can be attached to the loader body 1100. In an embodiment, the top loader bay 1200 can be coupled to a handle 1400 at a first portion of the top loader bay 1200 (such as a bottom portion). The top loader bay 1200 can further be coupled to a first sliding member 1510 of a first slide 1500 at a second portion (such as a top portion).

The first slide 1500 can include the first sliding member 1510 and a first track 1520. The first track 1520 can be affixed onto the loader body 1100 through appropriate means such as through one or more fasteners. The first sliding member 1510 can slidingly engage the first track 1520. The first slide 1500 can be a slide system such as a ball bearing slides, roller slides, epoxy slides, or other suitable slide systems.

The handle 1400 can include a handle arm 1410 and a top loader bay arm 1420. The handle arm 1410 can be coupled to the top loader bay arm 1420 through a joint 1430. In some embodiments, the handle arm 1410 can pivot around the joint 1430 such that the handle arm 1410 can move in an upward and/or downward motion.

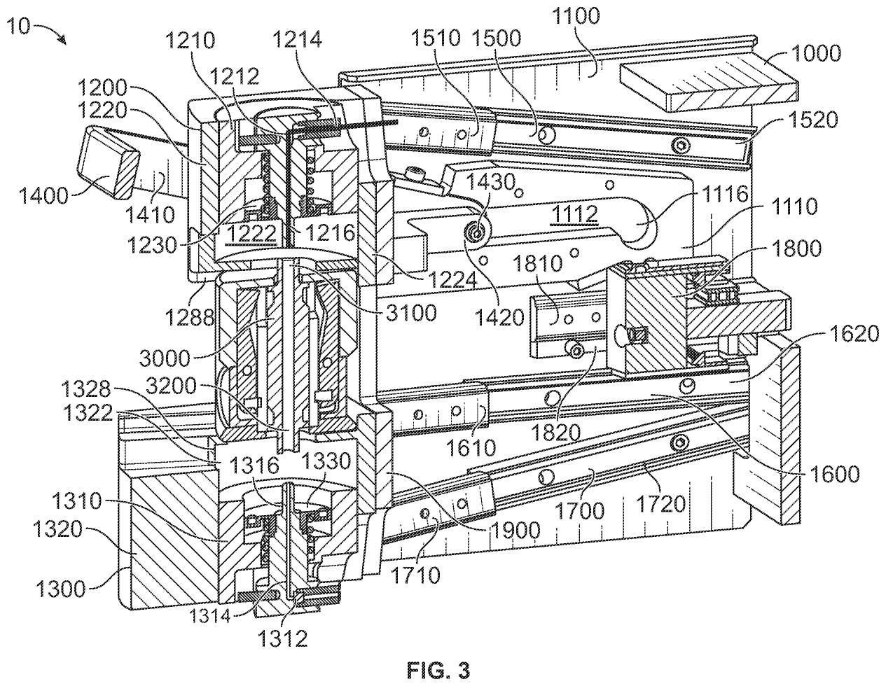

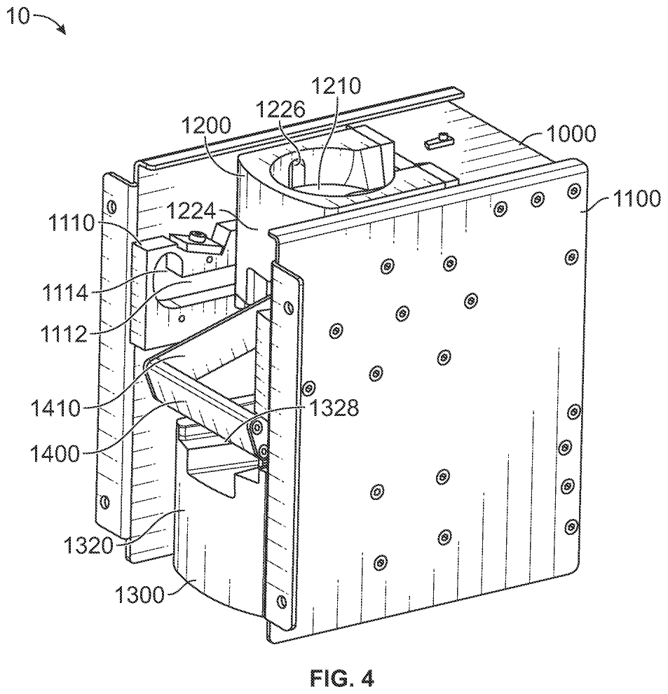

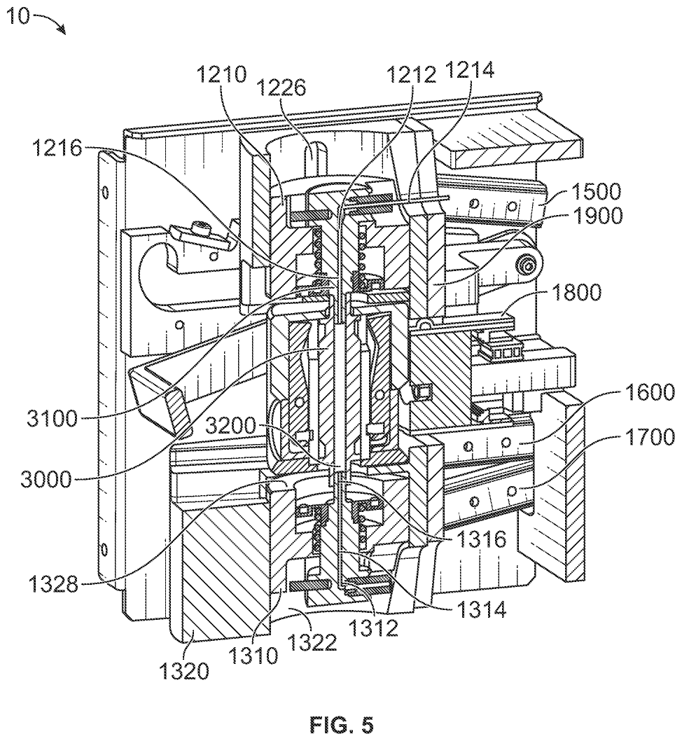

The handle 1400 can further slidingly engage a handle track 1110 on the loader body 1100. The handle track 1110 can be attached to the loader body 1100 through appropriate means such as through one or more fasteners. As best shown in FIGS. 3-5, the handle track 1110 can guide the handle 1400 to move in both a lateral direction (such as inwardly and outwardly with respect to the loader 1000) and a vertical direction (such as upwardly and downwardly with respect to the loader 1000). In some embodiments shown in FIGS. 3-5, the handle track 1110 can include a lateral portion 1112, a first vertical portion 1114, and a second vertical portion 1116. The first vertical portion 1114 can be proximal to a front side of the loader 1000 and extends upwardly, such that when the handle 1400 traverses the first vertical portion 1114, the top loader bay 1200 can be lifted upwardly away from the bottom loader bay 1300 (i.e., a first position). The second vertical portion 1116 can be proximal to a back side of the loader 1000 and extends downwardly, such that when the handle 1400 traverses the second vertical portion 1116, the top loader bay 1200 can be pressed downwardly toward to bottom loader bay 1300 (i.e., a second position). Thus, the loader 1000 can have a first configuration (i.e., when the handle 1400 is pulled fully outward and upward as shown in FIGS. 1-3), a second configuration (i.e., when the handle 1400 is pushed fully inward and downward as shown in FIGS. 4-5), and intermediary configurations (i.e., when the handle 1400 is traversing from the first position to the second position).

The top loader bay 1200 can include a first inner component 1210 and a first outer component 1220. The first inner component 1210 can have a generally rectangular or cylindrical shape and be positioned inside a first bore 1222 of the first outer component 1220 similar to a piston.

The first inner component 1210 can include a top fluid path 1212 that includes a top conduit 1214 and a top protrusion 1216. In some embodiments, the top protrusion 1216 can be inserted into a first end 3100 of the cartridge 3000 when the loader 1000 is in the second configuration (see FIG. 5). On the flip side, while the loader 1000 is in the first configuration, the top protrusion 1216 can be separated from the first end 3100 of the cartridge 3000 (see FIG. 3).

As the handle 1400 traverses from the first position to the second position, the top protrusion 1216 can be inserted into the first end 3100 of the cartridge 3000 until the top protrusion 1216 is fully inserted into the first end 3100 of the cartridge 3000 when the handle 1400 reaches the second position. Reversely, as the handle 1400 traverses from the second position to the first position, the top protrusion 1216 can be pull away from the first end 3100 of the cartridge 3000.

The first outer component 1220 can include a first side wall 1224 that forms the first bore 1222. The first side wall 1224 can include a first channel 1226. The first channel 1226 can allow the first inner component 1210 to be connected to the first slide 1500. The first slide 1500 can be mounted on the loader body 1100 at an angle toward a center axis such that in the first configuration, the first inner component 1210 can be proximal to a first end (such as the top end) of the first bore 1222 (see FIG. 3), whereas in the second configuration, the first inner component 1210 can be proximal to a second end (such as the bottom end) of the first bore 1222 (see FIG. 5).

The first outer component 1220 can further be coupled to the first handle 1400, such that the first outer component 1220 can be lifted away from the bottom loader bay 1300 when in the first configuration, and can be pushed closer to the bottom loader bay 1300 when in the second configuration.

The first outer component 1220 can further include a first receiving portion 1228 positioned toward an end of the top loader bay 1200 proximal to the bottom loader bay 1300. The first receiving portion 1228 can have a shape or profile that corresponds with a shape or profile of the cartridge shield 2000 such that a portion of the cartridge shield 2000 can be received in the first receiving portion 1228.

One or more first magnets 1230 can be provided on and/or in the top loader bay 1200. The first magnet 1230 can hold the cartridge shield 2000 in place when the cartridge shield 2000 is inserted into the holder 1000.

The bottom loader bay 1300 can be coupled to a second sliding member 1610 of a second slide 1600 and a third sliding member 1710 of a third slide 1700. The second slide 1600 can include the second sliding member 1610 and a second track 1620. Likewise, the third slide 1700 can include the third sliding member 1710 and a third track 1720.

The second track 1620 and the third track 1720 can each be affixed onto the loader body 1100 through appropriate means such as through one or more fasteners. The second sliding member 1610 can slidingly engage the second track 1620 and the third sliding member 1710 can slidingly engage the third track 1720. The second slide 1600 and the third slide 1700 can each be a slide system such as a ball bearing slides, roller slides, epoxy slides, or other suitable slide systems.

The bottom loader bay 1300 can include a second inner component 1310 and a second outer component 1320. The second inner component 1310 can have a generally rectangular or cylindrical shape and be positioned inside a second bore 1322 of the second outer component 1320 similar to a piston.

The second inner component 1310 can include a bottom fluid path 1312 that includes a bottom conduit 1314 and a bottom protrusion 1316. In some embodiments, the bottom protrusion 1316 can be inserted into a second end 3200 of the cartridge 3000 when the loader 1000 is in the second configuration (see FIG. 5). On the flip side, while the loader 1000 is in the first configuration, the bottom protrusion 1316 can be separated from the second end 3200 of the cartridge 3000 (see FIG. 3).

As the handle 1400 traverses from the first position to the second position, the bottom protrusion 1316 can be inserted into the second end 3200 of the cartridge 3000 until the bottom protrusion 1316 is fully inserted into the second end 3200 of the cartridge 3000 when the handle 1400 reaches the second position. Reversely, as the handle 1400 traverses from the second position to the first position, the bottom protrusion 1316 can be pull away from the second end 3200 of the cartridge 3000.

The second outer component 1320 can include a second side wall 1324 that forms the second bore 1322. The second side wall 1324 can include a second channel 1326. The second channel 1326 can allow the second inner component 1310 to be connected to the third slide 1700. The third slide 1700 can be mounted on the loader body 1100 at an angle toward a center axis such that in the first configuration, the third inner component 1310 can be proximal to a first end (such as the bottom end) of the first bore 1322 (see FIG. 3), whereas in the second configuration, the third inner component 1310 can be proximal to a second end (such as the top end) of the third bore 1322 (see FIG. 5).

The second outer component 1320 can further be coupled to the second slide 1600, such that the second outer component 1320 is pulled toward the front side of the loader 1000 when in the first configuration, and is pushed toward the back side of the loader 1000 when in the second configuration. In some embodiments, the second outer component 1320 can travel laterally inward and outward with respect to the loader 1000, while the second inner component 1310 can travel diagonally (i.e., both laterally and vertically), inward and outward, as well as upward and downward, with respect to the loader 1000.

The second outer component 1320 can further include a second receiving portion 1328 positioned proximal to an end of the bottom loader bay 1300 proximal to the top loader bay 1200. The second receiving portion 1328 can have a shape or profile that corresponds with a shape or profile of the cartridge shield 2000 such that a portion of the cartridge shield 2000 can be received in the second receiving portion 1328.

It can be appreciated although in some embodiments, the bottom loader bay 1300 does not include a handle, the bottom loader bay 1300 can nonetheless be moved due to friction when the cartridge shield 2000 is loaded into the loader 1000. Thus, when the cartridge shield 2000 is loaded into the loader 1000, the second inner component 1310 and the second outer component 1320 of the bottom loader bay 1300 can move in step with the second slide 1600 and the third slide 1700, respectively, when the handle 1400 is manipulated. Alternatively, one or more handles can also be provided on the bottom loader bay 1300 for more maneuverability.

One or more first magnets 1230 can be provided on and/or in the top loader bay 1200. The first magnet 1230 can hold the cartridge shield 2000 in place when the cartridge shield 2000 is inserted into the holder 1000. Likewise, one or more second magnets 1330 can be provided on and/or in the bottom loader bay 1300 for similar purposes.

The loader 1000 can further include a cleaning assembly 1800. In some embodiments, the cleaning assembly 1800 can be an ultraviolet (UV) cleaning assembly positioned proximal to the back side of the loader 1000. The cleaning assembly 1800 can be configured to automatically disinfect the loader 1000 when the loader 1000 is in its second configuration without a cartridge shield 2000 inserted. The cleaning assembly 1800 can be coupled to a fourth slide 1810 and a spring 1820, such that the cleaning assembly 1800 can be pushed out when the loader 1000 is in the second configuration with the cartridge shield 2000 inserted. The holder 1000 can also include a front plate 1900 that can be coupled to the handle 1400, the top loader bay 1200, and the bottom loader bay 1300.

The loader 1000 can include a plurality of sensors coupled to one or more processors and one or more non-transitory memories with machine executable programs stored thereon. Some sensors can be configured to detect the presence of a cartridge shield 2000. Some sensors can be configured to detect whether the cartridge shield 2000 is inserted properly. Some sensors can be configured to detect the cleaning assembly 1800. Some sensors can be configured to detect the presence of the cartridge 3000. Some sensors can be configured to detect that an elution has been started or a progress thereof. Certainly, sensors can be configured to detect additional conditions and parameters, and are within the scope of this disclosure.

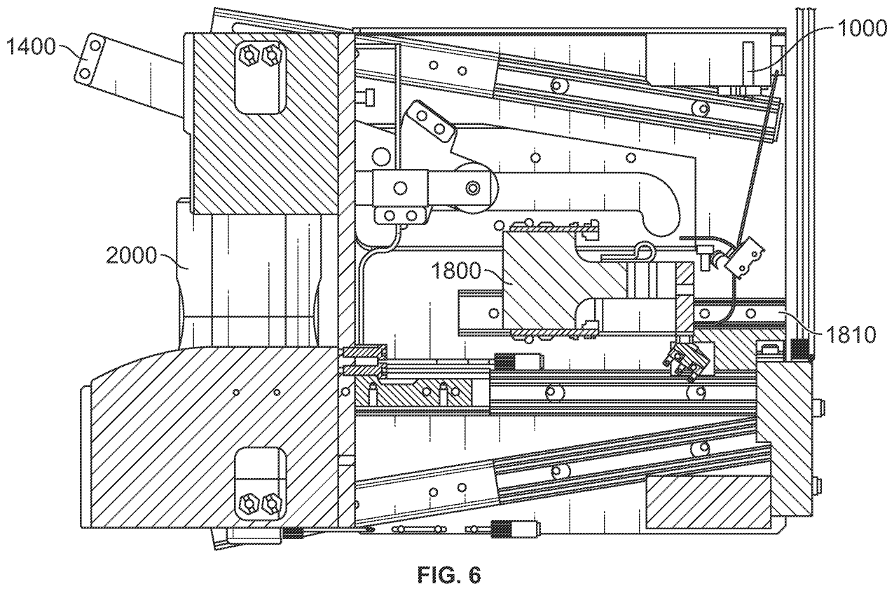

Referring to FIGS. 6-9, which illustrate the cleaning assembly 1800 according to some embodiments in more detail. As explained previously, the cleaning assembly 1800 can be mounted on the fourth slide 1810 and be coupled with the spring 1820. Thus, referring to FIG. 6, when the handle 1400 in the first position (i.e., when the loader 1000 is in the first configuration or “open”), the cleaning assembly 1800 can be in a forward position due to the spring 1820 not being compressed or extended depending on an installation of the spring 1820.

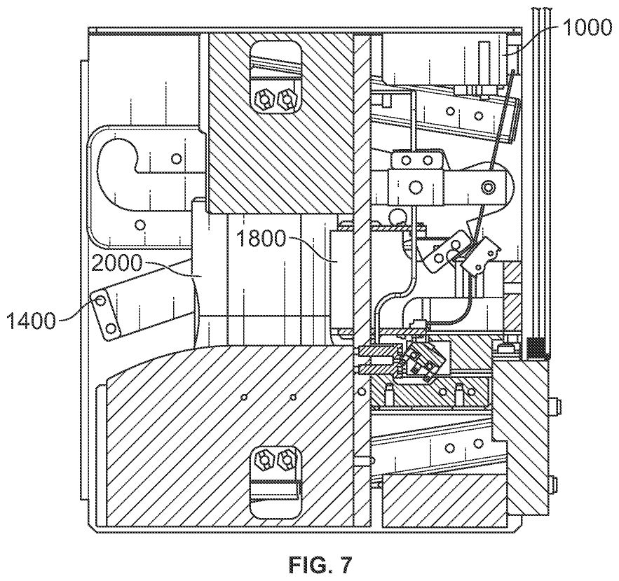

Referring to FIG. 7, when the handle 1400 is in the second position (i.e., the loader 1000 is in the second configuration 1000 or “closed”), if the cartridge shield 2000 is inserted into the loader 1000, the cleaning assembly 1800 is then displaced by the cartridge shield 2000, resulting in the cleaning assembly 1800 being pushed to a rearward position with the spring 1820 being compressed or extended.

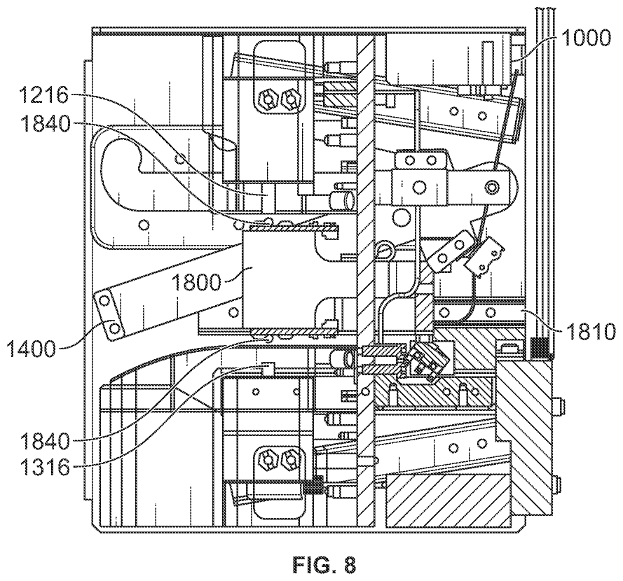

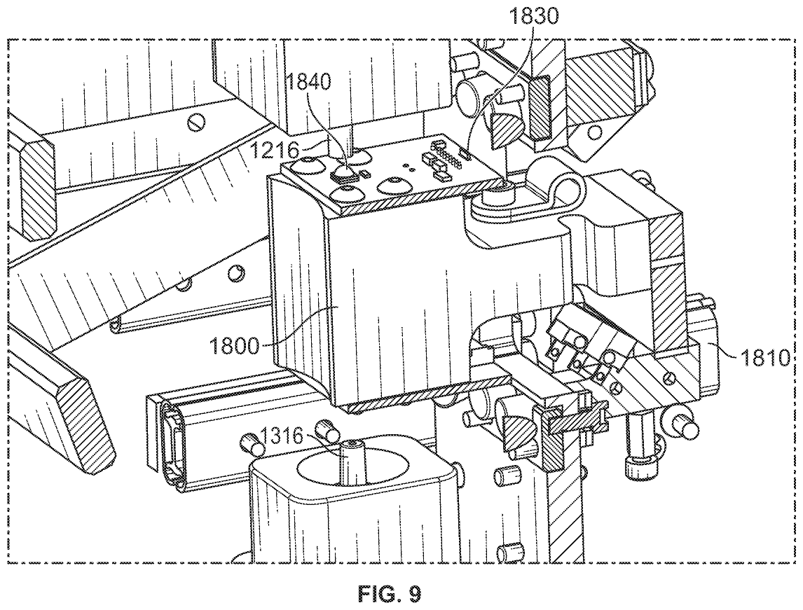

Referring to FIGS. 8 and 9, when the handle 1400 is in the second position (i.e., the loader 1000 is in the second configuration), but the cartridge shield 2000 is not inserted into the loader 1000, then the cleaning assembly 1800 can remain in the forward position since the spring 1820 is not compressed or extended. In this scenario, the cleaning assembly 1800 can be configured to turn on to disinfect the top protrusion 1216 of the top loader bay 1200 and/or the bottom protrusion 1316 of the bottom loader bay 1300.

A control system 1830 can be implemented to monitor electrical drive parameters such as current, voltage, impedance, temperature, wavelength, electrical power, and/or optical power to determine if the cleaning assembly 1800 is operating. The cleaning assembly 1800 can be positioned a distance away from the top loader bay 1200 and/or the bottom loader bay 1300 to ensure efficacy. However, in other embodiments, the cleaning assembly 1800 can be provided in a close proximity to the top loader bay 1200 and/or the bottom loader bay 1300, or directly or indirectly contact the top loader bay 1200 and/or the bottom loader bay 1300, such and other variations are also contemplated herein.

The cleaning assembly 1800 can be configured to stays on for a predetermined period of time. The control system 1830 can further be configured to detect whether the cleaning assembly 1800 is in a proper position to disinfect. In some embodiments, the cleaning assembly 1800 can include one or more UV light-emitting diodes 1840 (UV LEDs) that can be concentrically aligned and incident perpendicular with the top protrusion 1216 of the top loader bay 1200 and/or the bottom protrusion 1316 of the bottom loader bay 1300. Additional UV LEDs 1840 can also be provided to disinfect additional portions of the top loader bay 1200 and/or the bottom loader bay 1300. Of course, the cleaning assembly 1800 can also be used to disinfect additional aspect of the loader 1000, and such variations are also contemplated.

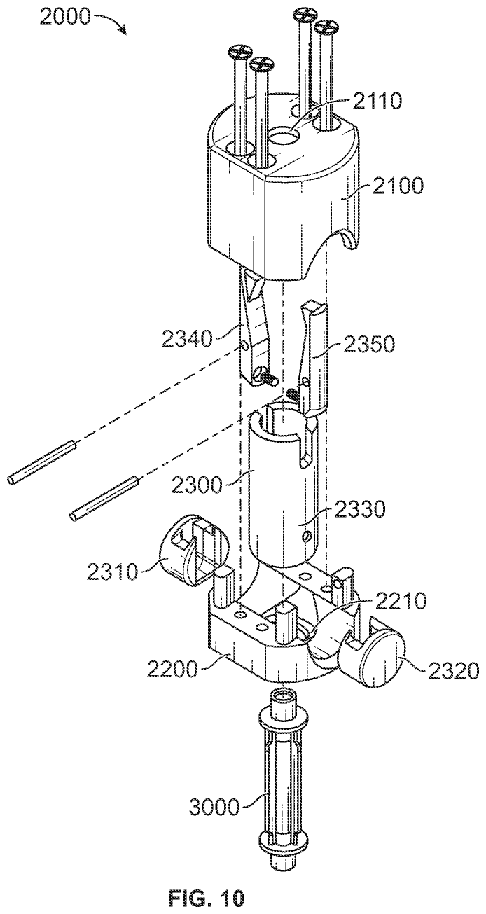

Referring to FIGS. 10 and 11, the cartridge shield 2000 can include a first shield body 2100 and a second shield body 2200. The first shield body 2100 and the second shield body 2200 can be affixed to each other through one or more fasteners.

The first shield body 2100 can include a first opening 2110 positioned centrally on a top surface 2120. Similarly, the second shield body 2200 can include a second opening 2210 positioned centrally on a bottom surface 2220. When the cartridge 3000 is inserted into the cartridge shield 2000, the first end 3100 of the cartridge 3000 can protrude out of the first opening 2110 and the second end 3200 of the cartridge 3000 can protrude out of the second opening 2210.

The cartridge shield 2000 can further include a holding mechanism 2300 positioned between the first shield body 2100 and the second shield body 2200. In some embodiments shown in FIGS. 10 and 11, the holding mechanism 2300 can be positioned proximal to a bottom end of the cartridge shield 2000. Alternatively, the holding mechanism 2300 can be positioned proximal to a top end, or the middle of the cartridge shield 2000. The holding mechanism 2300 can include a first button 2310, a second button 2320, a holding tube 2330, a first holding arm 2340, and a second holding arm 2350.

The first button 2310 can engage a first side of the holding tube 2330 and the first holding arm 2340. The second button 2320 can engage a second side of the holding tube 2330 opposite and the second holding arm 2350. The first holding arm 2340 and the second holding arm 2350 can be spring loaded or otherwise manipulable such that when the first button 2310 and the second button 2320 are manipulated (such as when pressed) by a user, the cartridge 3000 can be released from the holding tube 2330. Likewise, the cartridge 3000 can be inserted into the holding tube 2330.

The cartridge 3000 can be made out of a suitable soft material similar to a dropper or a Pasteur pipette. The cartridge 3000 can include the first end 3100 and the second end 3200 opposite from the first end 3100. The cartridge 3000 can be used to contain a radionuclide in the separation process.

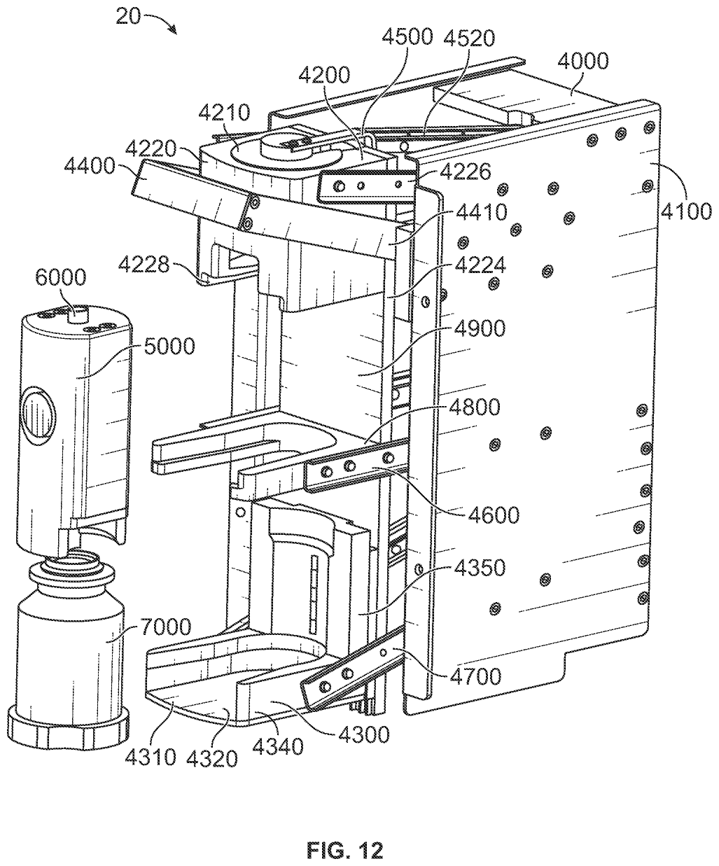

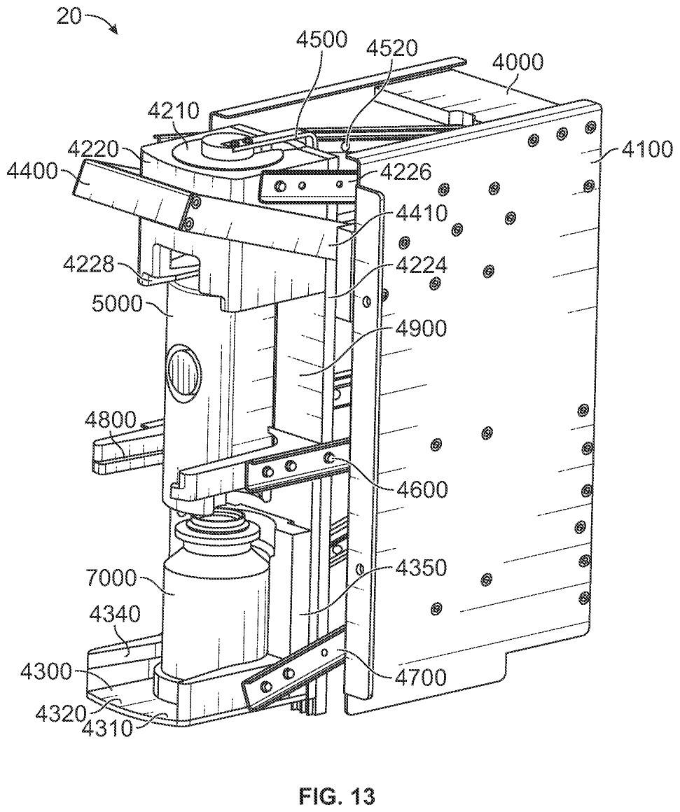

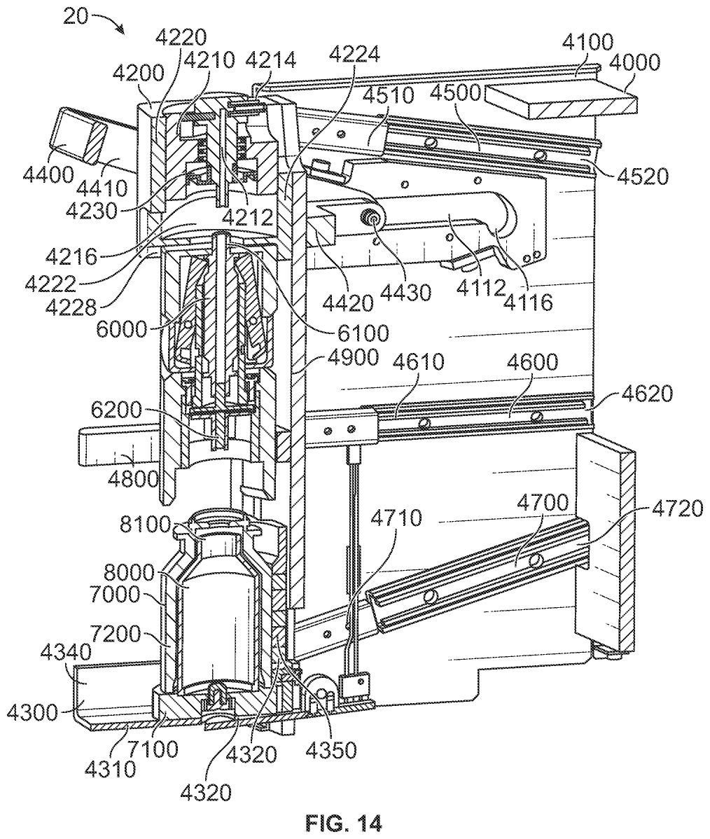

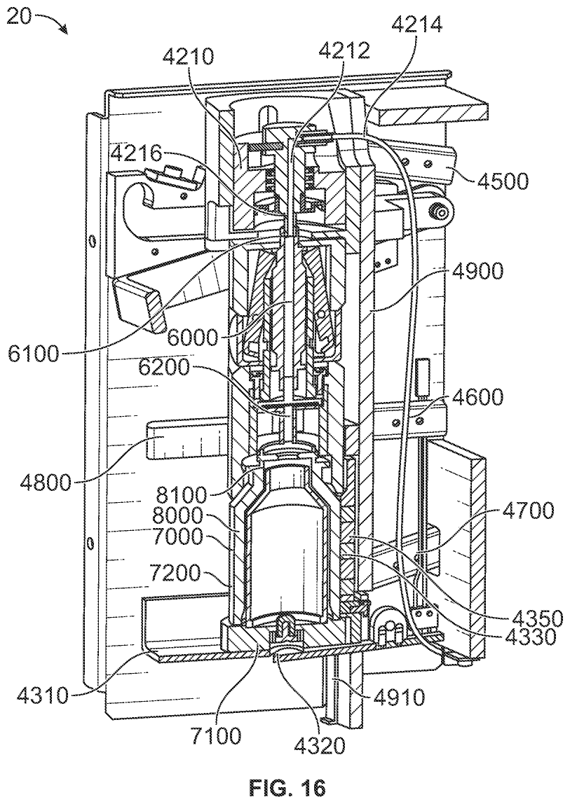

As shown in FIGS. 12-16, additional embodiments of this disclosure include a system 20 that includes a loader 4000 and a cartridge shield 5000. In some embodiments, the loader 4000 can be used in conjunction with a cartridge 6000 (such as a product cartridge) held in the cartridge shield 5000 (see FIG. 18) and a vial 8000 held in a vial shield 7000 (see FIGS. 14 and 16).

Referring to FIGS. 12-16, the loader 4000 can include a loader body 4100. A top loader bay 4200 and a bottom loader bay 4300 can be attached to the loader body 4100. The top loader bay 4200 can function substantially similar to the top loader bay 1200 of loader 1000 described above. In embodiments shown in FIGS. 12-16, the top loader bay 4200 can be coupled to a handle 4400 at a first portion of the top loader bay 4200 (such as a bottom portion). The top loader bay 4200 can further be coupled to a first sliding member 4510 of a first slide 4500 at a second portion (such as a top portion).

The first slide 4500 can include the first sliding member 4510 and a first track 4520. The first track 4520 can be affixed onto the loader body 4100 through appropriate means such as through one or more fasteners. The first sliding member 4510 can slidingly engage the first track 4520. The first slide 4500 can be a slide system such as a ball bearing slides, roller slides, epoxy slides, or other suitable slide systems.

The handle 4400 can include a handle arm 4410 and a top loader bay arm 4420. The handle arm 4410 can be coupled to the top loader bay arm 4420 through a joint 4430. In some embodiments, the handle arm 4410 can pivot around the joint 4430 such that the handle arm 4410 can move in an upward or downward motion.

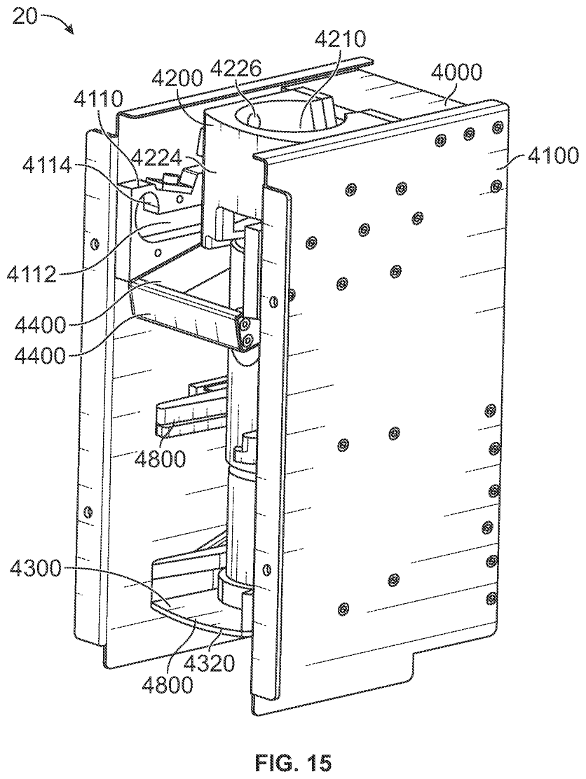

The handle 4400 can further slidingly engage a handle track 4110 on the loader body 4100. The handle track 4110 be attached to the loader body 4100 through appropriate means such as through one or more fasteners. As best shown in FIGS. 14-16, the handle track 4110 can guide the handle 4400 to move in both a lateral direction (such an inwardly and outwardly with respect to the loader 4000) and a vertical direction (such as upwardly and downwardly with respect to the loader 4000). In some embodiments shown in FIGS. 14-16, the handle track 4110 can include a lateral portion 4112, a first vertical portion 4114, and a second vertical portion 4116. The first vertical portion 4114 can be proximal to a front side of the loader 4000 and extends upwardly, such that when the handle 4400 traverses the first vertical portion 4114, the top loader bay 4200 can be lifted upwardly away from the bottom loader bay 4300 (i.e., a first position). The second vertical portion 4116 can be proximal to a back side of the loader 4000 and extends downwardly, such that when the handle 4400 traverses the second vertical portion 4116, the top loader bay 4200 can be pressed downwardly toward to bottom loader bay 4300 (i.e., a second position). Thus, the loader 4000 can have a first configuration (i.e., when the handle 4400 is pulled fully outward and upward as shown in FIGS. 12-14), a second configuration (i.e., when the handle 4400 is pushed fully inward and downward as shown in FIGS. 15-16), and intermediary configurations (i.e., when the handle 4400 is traversing from the first position to the second position).

The top loader bay 4200 can include a first inner component 4210 and a first outer component 4220. The first inner component 4210 can have a generally rectangular or cylindrical shape and be positioned inside a first bore 4222 of the first outer component 4220 similar to a piston.

The first inner component 4210 can include a top fluid path 4212 that includes a top conduit 4214 and a top protrusion 4216. In some embodiments, the top protrusion 4216 can be inserted into a first end 6100 of the cartridge 6000 when the loader 4000 is in the second configuration (see FIG. 16). On the flip side, while the loader 4000 is in the first configuration, the top protrusion 4216 can be separated from the first end 6100 of the cartridge 6000 (see FIG. 14).

As the handle 4400 traverses from the first position to the second position, the top protrusion 4216 can be inserted into the first end 6100 of the cartridge 6000 until the top protrusion 4216 is fully inserted into the first end 6100 of the cartridge 6000 when the handle 4400 reaches the second position. Reversely, as the handle 4400 traverses from the second position to the first position, the top protrusion 4216 can be pull away from the first end 6100 of the cartridge 6000.

The first outer component 4220 can include a first side wall 4224 that forms the first bore 4222. The first side wall 4224 can include a first channel 4226. The first channel 4226 can allow the first inner component 4210 to be connected to the first slide 4500. The first slide 4500 can be mounted on the loader body 4100 at an angle toward a center axis such that in the first configuration, the first inner component 4210 can be proximal to a first end (such as the top end) of the first bore 4222 (see FIG. 14), whereas in the second configuration, the first inner component 4210 can be proximal to a second end (such as the bottom end) of the first bore 4222 (see FIG. 16).

The first outer component 4220 can further be coupled to the first handle 4400, such that the first outer component 4220 can be lifted away from the bottom loader bay 4300 when in the first configuration, and can pushed closer to the bottom loader bay 4300 when in the second configuration.

The first outer component 4220 can further include a first receiving portion 4228 positioned toward an end of the top loader bay 4200 proximal to the bottom loader bay 4300. The first receiving portion 4228 can have a shape or profile that corresponds with a shape or profile of the cartridge shield 5000 such that a portion of the cartridge shield 5000 can be received in the first receiving portion 4228.

One or more first magnets 4230 can be provided on and/or in the top loader bay 4200. The first magnet 4230 can hold the cartridge shield 5000 in place when the cartridge shield 5000 is inserted into the holder 4000.

The loader 4000 can further include a cartridge shield holder 4800 coupled to a second slide 4600. The second slide 4600 can include the second sliding member 4610 and a second track 4620. The second slide 4600 can be affixed onto the loader body 4100 through appropriate means such as through one or more fasteners. The second sliding member 4610 can slidingly engage the second track 4620. The second slide 4600 can be a slide system such as a ball bearing slides, roller slides, epoxy slides, or other suitable slide systems.

The second slide 4600 can be configured to allow the cartridge shield holder 4800 to traverse from a position proximal to the front of the loader 4000 when the loader 4000 is in the first configuration to a position proximal to the back of the loader 4000 when the loader 4000 in in the second configuration.

The bottom loader bay 4300 can be coupled to a third slide 4700. The third slide 4700 can include a third sliding member 4710 and a third track 4720. The third slide 4700 can be affixed onto the loader body 4100 through appropriate means such as through one or more fasteners. The third sliding member 4710 can slidingly engage the third track 4720. The third slide 4700 can be a slide system such as a ball bearing slides, roller slides, epoxy slides, or other suitable slide systems.

The third slide 4700 can be mounted on the loader body 4100 at an angle toward a center axis such that in the first configuration, the bottom loader bay 4300 can be proximal to a first side (such as the bottom side) and the front side of the loader 4000 (see FIG. 14, i.e., forward and lower), whereas in the second configuration, the bottom loader bay 4300 can be elevated relative to the first end and toward the back side of the loader (see FIG. 16, i.e., rearward and higher).

The bottom loader bay 4300 can further include a second receiving portion 4310. The second receiving portion 4310 can have a shape or profile that corresponds with a shape or profile of the vial shield 7000 such that a portion of the vial shield 7000 can be received in the second receiving portion 4310.

One or more second magnets 4330 can be provided on and/or in the top loader bay 4200. The second magnets 4330 can hold the vial shield 7000 in place when the vial shield 7000 is inserted into the holder 4000.

The second receiving portion 4310 can be formed from a base wall 4320, a second side wall 4340 protruding from the base wall 4320 that is generally perpendicular to the base wall 4320, and a third side wall 4350 extending from the second side wall 4340. The second side wall 4340 can have a generally U-Shape internal profile such that the second side wall 4340 can hold a base portion 7100 of the vial shield 7000. The third side wall 4350 can have an internal profile that is generally concaved to fit an outer profile of a body portion 7200 of the vial shield 7000.

The holder 4000 can also include a front plate 4900 that can be coupled to the handle 4400, the top loader bay 4200, the cartridge shield holder 4800 and the bottom loader bay 4300. The bottom loader bay 4300 can be slidingly engaged to the front plate 4900 through a bottom loader bay slide 4910 (see FIG. 16).

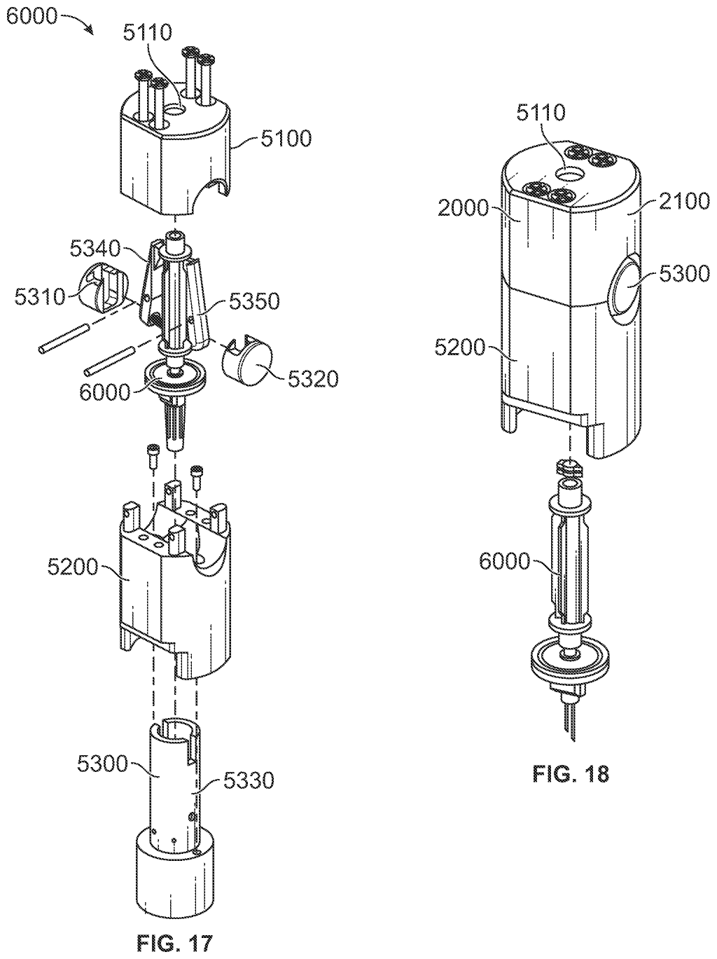

Referring to FIGS. 17 and 18, a cartridge shield 5000 can be structurally similar to the cartridge shield 2000 of FIGS. 11 and 12. The cartridge shield 5000 can include a first shield body 5100 and a second shield body 5200. The first shield body 5100 and the second shield body 5200 can be affixed to each other through one or more fasteners.

The first shield body 5100 can include a first opening 5110 positioned centrally on a top surface 5120. When the cartridge 6000 is inserted into the cartridge shield 5000, the first end 6100 of the cartridge 6000 can protrude out of the first opening 5110.

The cartridge shield 5000 can further include a holding mechanism 5300 positioned between the first shield body 5100 and the second shield body 5200. The holding mechanism 5300 can include a first button 5310, a second button 5320, a holding tube 5330, a first holding arm 5340, and a second holding arm 5350.

The first button 5310 can engage a first side of the holding tube 5330 and the first holding arm 5340. The second button 5320 can engage a second side of the holding tube 5330 opposite and the second holding arm 5350. The holding tube 5330 can be affixed to the second shield body 5200 through one or more fasteners. The first holding arm 5340 and the second holding arm 5350 can be spring loaded or otherwise manipulable such that when the first button 5310 and the second button 5320 are manipulated (such as when pressed) by a user, the cartridge 6000 can be released from the holding tube 8330. Likewise, the cartridge 6000 can be inserted into the holding tube 8330.

The cartridge 6000 can be made out of a suitable soft material similar to a dropper or a Pasteur pipette. The cartridge 6000 can include the first end 6100 and a second end 6200 opposite from the first end 6100. The cartridge 6000 can be used to contain a product such as ^{9m}Tc.

The second end 6200 of the cartridge 6000 can be aligned with a vial septa 8100 of the vial 8000 as the loader 4000 transitions from its first configuration to the second configuration. As the loader 4000 approaches the second configuration, the second end 6200 of the can pierce the vial septa 8100 (see FIG. 16).

The vial shield 7000 can include the base portion 7100 and the body portion 7200. The body portion can have a inner shape that is similar to a shape of the vial 8000 to be held therein. The body portion can include an opening 7210 on a side (such as its top side) opposite from the base portion 7100 that allows the second end 6200 of the cartridge 6000 to protrude therethrough when the loader 4000 is in the second configuration, thus piercing the vial septa 8100.

The loader 4000 can include a plurality of sensors coupled to one or more processors and one or more non-transitory memories with machine executable programs stored thereon. Some sensors can be configured to detect the presence of a cartridge shield 5000. Some sensors can be configured to detect whether the cartridge shield 5000 is inserted properly. Some sensors can be configured to detect the vial shield 7000. Some sensors can be configured to detect that an elution has been started or a progress thereof. Certainly, sensors can be configured to detect addition conditions and parameters, and are within the scope of this disclosure.

Specific embodiments of a cartridge loader according to this disclosure have been described for the purpose of illustrating the manner in which the invention can be made and used. It should be understood that the implementation of other variations and modifications of this invention and its different aspects will be apparent to one skilled in the art, and that this invention is not limited by the specific embodiments described. Features described in one embodiment can be implemented in other embodiments. The subject disclosure is understood to encompass this disclosure and any and all modifications, variations, or equivalents that fall within the spirit and scope of the basic underlying principles disclosed and claimed herein.

What is claimed is:

1. A cartridge loader comprising:
a loader body;
a top loader bay slidingly engaged to the loader body; and
a bottom loader bay slidingly engaged to the loader body,
wherein the loader body is configured to receive a cartridge shield with a cartridge provided therein between the top loader bay and the bottom loader bay, and
wherein the cartridge loader can be manipulated between a first configuration and a second configuration.

2. The cartridge loader of claim 1 further comprising:
a handle track provided on the loader body; and
a handle slidingly engaging the handle track such that the handle is configured to move in both a lateral direction and a vertical direction with respect to the loader body.

3. The cartridge loader of claim 2, wherein the handle track further comprising a horizontal portion and a vertical portion.

4. The cartridge loader of claim 2, wherein the top loader bay further comprising a first inner component and a first outer component, wherein the first inner component is slidingly received within a first bore of the first outer component.

5. The cartridge loader of claim 4, wherein the first inner component further comprising a top protrusion configured to be inserted into a first end of the cartridge when the cartridge loader is in the second configuration.

6. The cartridge loader of claim 5, wherein the top protrusion is removed from the cartridge when the cartridge loader is in the first configuration.

7. The cartridge loader of claim 4, wherein the first outer component further comprising a channel such that the first inner component can be coupled to a first slide through the channel.

8. The cartridge loader of claim 7, wherein the first outer component is coupled to the handle such that the first outer component can be lifted away from the bottom loader bay when the cartridge loader is in the first configuration, and the first outer component can be pushed toward the bottom loader bay when the cartridge loader is in the second configuration.

9. The cartridge loader of claim 4, wherein the first outer component further comprising a first receiving portion for receiving a portion of the cartridge shield.

10. The cartridge loader of claim 1, wherein the top loader bay further comprising a magnet for holding the cartridge shield.

11. The cartridge loader of claim 4, wherein the bottom loader bay further comprising a second inner component and a second outer component, wherein the second inner component is slidingly received within a second bore of the second outer component.

12. The cartridge loader of claim 11, wherein the second inner component further comprising a bottom protrusion configured to be inserted into a second end of the cartridge when the cartridge loader is in the second configuration.

13. The cartridge loader of claim 12, wherein the bottom protrusion is removed from the cartridge when the cartridge loader is in the first configuration.

14. The cartridge loader of claim 11, wherein the second outer component further comprising a channel such that the second inner component can be coupled to a second slide through the channel.

15. The cartridge loader of claim 11, wherein the second outer component further comprising a second receiving portion for receiving a portion of the cartridge shield.

16. The cartridge loader of claim 1, wherein the bottom loader bay further comprising a magnet for holding the cartridge shield.

17. The cartridge loader of claim 1 further comprising:
a cleaning assembly slidingly coupled to the loader body,
wherein when the cartridge loader is in the second configuration without receiving the cartridge shield, the cleaning assembly is configured to disinfect the top loader bay and the bottom loader bay.

18. The cartridge loader of claim 17, wherein the cleaning assembly further comprising one or more ultraviolet light-emitting diodes (UV LEDs) positioned to disinfect the top loader bay and the bottom loader bay.

19. The cartridge loader of claim 1, wherein the bottom loader bay is further configured to hold a vial shield with a vial provided therein.

20. The cartridge loader of claim 1, wherein the bottom loader bay is configured to slide between a first position and a second position due to friction caused by the cartridge shield as the cartridge loader transitions from the first configuration to the second configuration.
